(19) United States
(12) Patent Application Publication
(10) Pub. No.: US 2008/0319534 A1
(43) Pub. Date: Dec. 25, 2008

(54) Stent With Improved Mechanical Properties

(75) Inventors: Matthew J. Birdsall, Santa Rosa, CA (US); Gianfranco Pellegrini, Santa Rosa, CA (US); Justin Goshgarian, Santa Rosa, CA (US); Jeffrey Allen, Santa Rosa, CA (US)

Correspondence Address: MEDTRONIC VASCULAR, INC.;IP LEGAL DEPARTMENT, 3576 UNOCAL PLACE, SANTA ROSA, CA, 95403, US

(73) Assignee: Medtronic Vascular, Inc., Santa Rosa, CA (US)

(21) Appl. No.: 11/767,308

(22) Filed: Jun. 22, 2007

(51) Int. Cl. A61F 2/06

(52) U.S. Cl. 623/1.22

(57) ABSTRACT

A stent includes a central portion having a first waveform. The first waveform is wrapped around a longitudinal axis of the stent at a pitch to define a plurality of helical turns. The stent also includes an end segment connected to one end of the central portion. The end segment has a second waveform that includes a plurality of struts and a plurality of crowns. Each of the plurality of struts has a different length so that peaks of the crowns that define an end of the stent lie within a plane that is substantially perpendicular to the longitudinal axis. Cross-sectional areas of the struts having different lengths vary so that the struts move substantially uniformly during radial contraction and/or radial expansion of the stent.

### BACKGROUND OF THE INVENTION

1. Field of the Invention

The present invention generally relates to stents. More particularly, the present invention relates to helical coil stents having improved mechanical properties.

2. Description of Related Art

Percutaneous transluminal angioplasty (PTCA) is used to open coronary arteries, which have been occluded by a build-up of cholesterol fats or atherosclerotic plaque. Typically, a guide catheter is inserted into a major artery in the groin and is passed to the heart, providing a conduit to the ostia of the coronary arteries from outside the body. A balloon catheter and guidewire are advanced through the guiding catheter and steered through the coronary vasculature to the site of therapy. The balloon at the distal end of the catheter is inflated, causing the site of the stenosis to widen. Dilation of the occlusion, however, can form flaps, fissures or dissections, which may threaten, reclosure of the dilated vessel. Implantation of a stent can provide support for such flaps and dissections and thereby prevent reclosure of the vessel. Reducing the possibility of restenosis after angioplasty may reduce the likelihood that a secondary angioplasty procedure or a surgical bypass operation will be needed.

A stent is typically a hollow, generally cylindrical device that is deployed in a body lumen from a radially contracted configuration into a radially expanded configuration, which allows it to contact and support the vessel wall. A plastically deformable stent can be implanted during an angioplasty procedure by using a balloon catheter bearing a compressed or “crimped” stent, which has been loaded onto the balloon. The stent radially expands as the balloon is inflated, forcing the stent into contact with the body lumen, thereby forming a support for the vessel wall. Deployment is effected after the stent has been introduced percutaneously, transported transluminally, and positioned at a desired location by means of the balloon catheter.

Stents may be formed from wire(s), may be cut from a tube, or may be cut from a sheet of material and then rolled into a tube-like structure. While some stents include a plurality of connected rings that are substantially parallel to each other and are oriented so that the ends of the rings are substantially perpendicular to a longitudinal axis of the stent, others include a helical coil that is wrapped around the longitudinal axis at a certain pitch.

Helical stents tend to have ends that are not perpendicular to the longitudinal axis due to the pitch of the helix. To square off the ends of a helical stent, the last turn at either end may include a waveform that includes waves of varying amplitudes. However, by varying the amplitudes of the waves, the stent may exhibit non-uniform behavior as the stent is crimped onto a balloon and/or expanded at the deployment site, due to different moments and bending forces being incurred by the different portions of the waveform. For example, during deployment of the stent, the ends of the stent may expand before the central portion of the stent, thereby causing a so-called “dog bone” effect, and the last turn at either end may expand non-uniformly due to the varying amplitudes of the waves contained therein.

It is desirable to provide a helical stent that has improved mechanical properties so that the stent may contract and expand more uniformly, and the “dog bone” effect during expansion may be substantially eliminated.

### SUMMARY OF THE INVENTION

It is an aspect of the present invention to provide a stent having improved mechanical properties so that the stent may be crimped and deployed more uniformly.

In an embodiment, a stent includes a central portion having a first waveform. The first waveform is wrapped around a longitudinal axis of the stent at a pitch to define a plurality of helical turns. The stent also includes an end segment connected to one end of the central portion. The end segment has a second waveform that includes a plurality of struts and a plurality of crowns. Each of the plurality of struts has a different length so that peaks of the crowns that define an end of the stent lie within a plane that is substantially perpendicular to the longitudinal axis. Cross-sectional areas of the struts having different lengths vary so that the struts move substantially uniformly during radial contraction and/or radial expansion of the stent.

In an embodiment, a stent includes a central portion having a first waveform formed by a continuous wire. The first waveform is wrapped about a longitudinal axis of the stent so as to form a helix. The stent also includes an end segment having a second waveform formed from a tube or sheet of material. The second waveform includes a plurality of struts and a plurality of crowns, each of the plurality of struts has a different length so that peaks of the crowns that define an end of the stent lie within a plane that is substantially perpendicular to the longitudinal axis. The stent further includes a first connector constructed and arranged to connect a first end of the second waveform to the central portion, and a second connector constructed and arranged to connect a second of the second waveform to the central portion.

In an embodiment, a method of manufacturing a stent includes forming a first waveform, wrapping the first waveform around a mandrel at a predetermined pitch to form a helical shape, and forming a second waveform. The second waveform has a plurality of undulations that decrease in amplitude and in cross-sectional area between a first end of the second waveform and a second end of the second waveform. The method further includes connecting the first end and the second end of the second waveform to the first waveform.

In an embodiment, a method of manufacturing a stent includes forming a first waveform and a second waveform from a solid piece of material. The first waveform has a first plurality of undulations disposed about a longitudinal axis at a pitch so as to form a helix, and the second waveform is connected to one end of the first waveform and has a second plurality of undulations that decrease in amplitude and in cross-sectional area between a first end of the second waveform and a second end of the second waveform.

These and other aspects and advantages of the invention will be apparent from the following description, the accompanying drawings, and the appended claims.

### BRIEF DESCRIPTION OF THE DRAWINGS

Embodiments of the present invention will now be described, by way of example only, with reference to the accompanying schematic drawings in which corresponding reference symbols indicate corresponding parts, and in which:

FIG. 1 illustrates a stent according to an embodiment of the present invention;

FIG. 2 illustrates a detailed view of an embodiment of a central portion of the stent of FIG. 1;

FIG. 3 illustrates a more detailed view of an embodiment of the central portion of the stent of FIG. 1;

FIG. 4 illustrates a detailed view of an embodiment of an end portion of the stent of FIG. 1 in an unrolled configuration; and

FIG. 5 illustrates a more detailed view of the end portion of FIG. 4.

### DETAILED DESCRIPTION OF EMBODIMENTS OF THE INVENTION

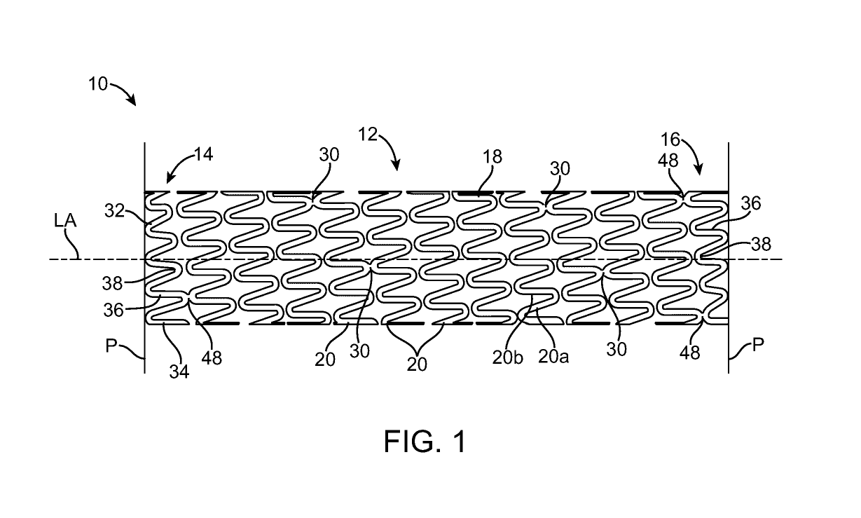

FIG. 1 illustrates a stent 10 according to an embodiment of the present invention. As illustrated, the stent 10 includes a central portion 12, a first end segment 14 that is connected to one end of the central portion 12, and a second end segment 16 that is connected to an opposite end of the central portion 12 as the first end segment 14. The stent 10 is generally cylindrical in shape and has a longitudinal axis LA extending through the center of the stent 10, as shown in FIG. 1.

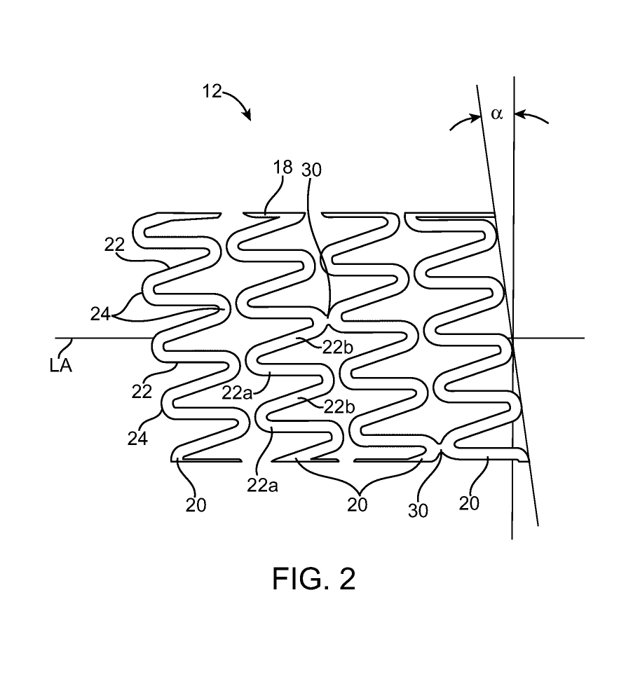

The central portion 12 of the stent, a portion of which is shown in greater detail in FIG. 2, is defined by a continuous waveform 18 that is wrapped around the longitudinal axis LA at a predetermined pitch a to form a helix having a plurality of helical turns 20. The continuous waveform 18 includes a plurality of struts 22 and a plurality of crowns 24 (or turns) that connect adjacent struts to each other. As illustrated, the struts 22 are substantially straight, although it is contemplated that in other embodiments, the struts may be slightly bent or have other shapes, such as a sinusoidal wave, for example. In some embodiments, the struts 22 may all be of substantially the same length, but in the illustrated embodiment, the struts 22 include longer struts 22a and shorter struts 22b. By having longer struts 22a and shorter struts 22b within the continuous waveform 20, the crowns 24 may be oriented substantially parallel to the longitudinal axis LA, while still maintaining the helix around the longitudinal axis, as illustrated in FIG. 1.

However, by varying the lengths of the struts 22 in the continuous waveform 20, different moments and bending forces may be created when the stent 10 radially contracts or expands, e.g. when the stent 10 is crimped onto a balloon catheter prior to delivery to the targeted site or when the stent 10 is expanded at the site during deployment. Different moments and bending forces that are created within the stent during contraction or expansion may cause the stent to contract or expand unevenly, which may not only result in an undesired shape, but may also create uneven stress within the stent, and may ultimately impede the performance of the stent. To compensate for the different moments and bending forces that are created by the struts 22 having different lengths, the cross-sectional areas of the struts 22 may be varied.

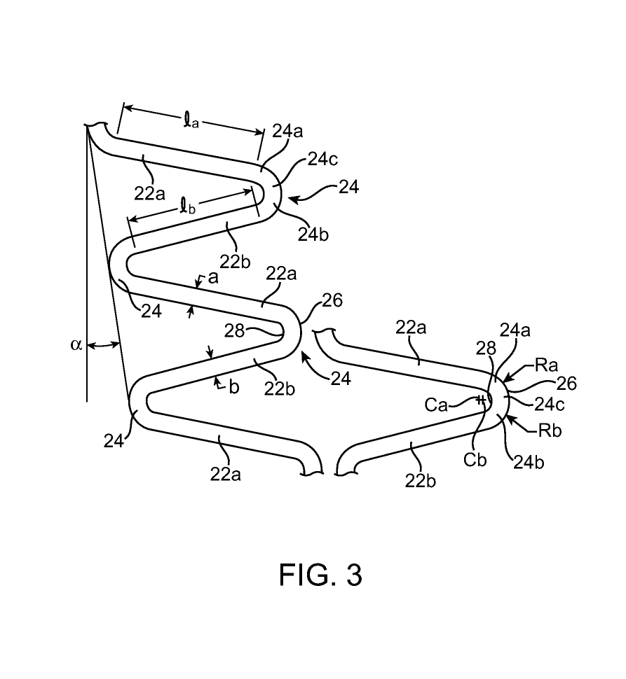

For example, as shown in FIG. 3, the longer strut 22a has a length lₐ and the shorter strut has a length l_{b}, which is less than the length lₐ. The longer strut 22a also has greater cross-sectional area than the shorter strut 22b. This is represented by the different widths of the struts shown in FIG. 3. For example, the longer strut 22a has a width ‘a’, and the shorter strut has a width ‘b’, which is less than width ‘a’, and the thickness of the longer strut 22a is the same as the thickness of the shorter strut 22b, so that the cross-sectional area of the longer strut 22a is greater than the cross-sectional area of the shorter strut 22b. Of course, the cross-sectional areas of a strut may be changed by altering the width and/or thickness of the strut if the strut has a substantially rectangular cross-section, or altering the diameter of the strut if the strut has a substantially circular cross-section, or altering the dimensions of the minor axis and major axis if the strut has an ellipsoidal cross-section. for example. The appropriate cross-sectional area of a strut may be calculated for the given length of the strut and the anticipated moments and bending forces that will be incurred by the strut during contraction and expansion of the stent. Because the longer strut 22a and the shorter strut 22b have different cross-sections and are joined by a crown 24, the crown 24 may also be shaped so that it transitions smoothly between the two different cross-sections, while still maintaining the appropriate level of mechanical integrity.

For example, as illustrated in FIG. 3, the crown 24 that connects the longer strut 22a to the shorter strut 22b is shaped so that the width of the portion of the crown 24 that is connected to the longer strut 22a, represented by 24a, has substantially the same width (a) as the longer strut 22a. Similarly, the portion of the crown 24 that is connected to the shorter strut 22b, represented by 24b, has substantially the same width as the width (b) of the shorter strut 22b. An intermediate portion 24c of the crown 24 that is in between the portions 24a and 24b, has a varying width that gradually transitions from width ‘a’ to width ‘b’. In an embodiment, the centers of radii of curvatures that define the outer curved surfaces of portions 24a and 24b of the crown 24 may be off-set to create the gradual transition from width a to width b. For example, as illustrated in FIG. 3, the crown 24 includes an outer surface 26 and an inner surface 28. The radius of the inner surface 28 may be constant, while the outer surface 26 may be defined by an outer radius Rₐ that has a center of curvature located at point Cₐ, and an outer radius R_{b} that has a center of curvature located at point C_{b}. As shown in FIG. 3, points Cₐ and C_{b} do not coincide and are offset from one another, so that the crown 24 has a varying width. The specific crown configuration depicted is provided as an example and is not intended to be limiting in any way.

The central portion 12 of the stent may be formed from a wire, or may be cut from a sheet or tube of material with a laser or etched from a sheet or tube of material with chemicals. In embodiments in which the central portion 12 is formed from a wire, the wire may be drawn down to appropriate cross-sections so that when the waveform 20 is formed, the appropriate struts have the appropriate cross-sectional areas and the corresponding crowns have the appropriate shapes for accommodating different moments and bending forces throughout the central portion 12 of the stent during radial contraction and/or expansion of the stent. In embodiments in which the central portion 12 is cut from a tube or sheet of material, the tool or method being used to cut the material may be programmed to shape the waveform 20 so that the struts 22 have the appropriate lengths and cross-sectional areas and the crowns 24 likewise have the appropriate shapes for handling the different moments and bending forces incurred by the struts 22 so that the central portion 12 will behave substantially uniformly during radial contraction and/or expansion of the stent.

The central portion 12 may be formed from any suitable material, including but not limited to stainless steel, iridium, platinum, gold, tungsten, tantalum, palladium, silver, niobium, zirconium, aluminum, copper, indium, ruthenium, molybdenum, niobium, tin, cobalt, nickel, zinc, iron, gallium, manganese, chromium, titanium, aluminum, vanadium, and carbon, as well as combinations, alloys, and/or laminations thereof. For example, the central portion 12 may be formed from a cobalt-chrome alloy, such as L605, a nickel-cobalt alloy having low titanium, such as MP35N®, Nitinol (nickel-titanium shape memory alloy), ABI (palladium-silver alloy), Elgiloy® (cobalt-chromium-nickel alloy), etc. It is also contemplated that the central portion may be formed from tantalum that is laminated with MP35N®, or from a drawn filled tube, such as DFT® manufactured by Fort Wayne Metals. The aforementioned materials and laminations are intended to be examples and are not intended to be limiting in any way.

As shown in FIG. 1, adjacent helical turns 20 may be connected with a plurality of connectors 30. The connectors 30 may include a weld, such as a spot weld, or in embodiments in which the central portion 12 is cut from a tube or sheet of material, the connectors 30 may be integrally formed with the crowns 24 of adjacent helical turns 20. In the illustrated embodiment, not every crown is connected to a crown of an adjacent helical turn 20. The connectors 30 may increase the longitudinal stiffness of the stent 10, while still allowing the stent 10 to be flexible as it is advanced to the targeted deployment site.

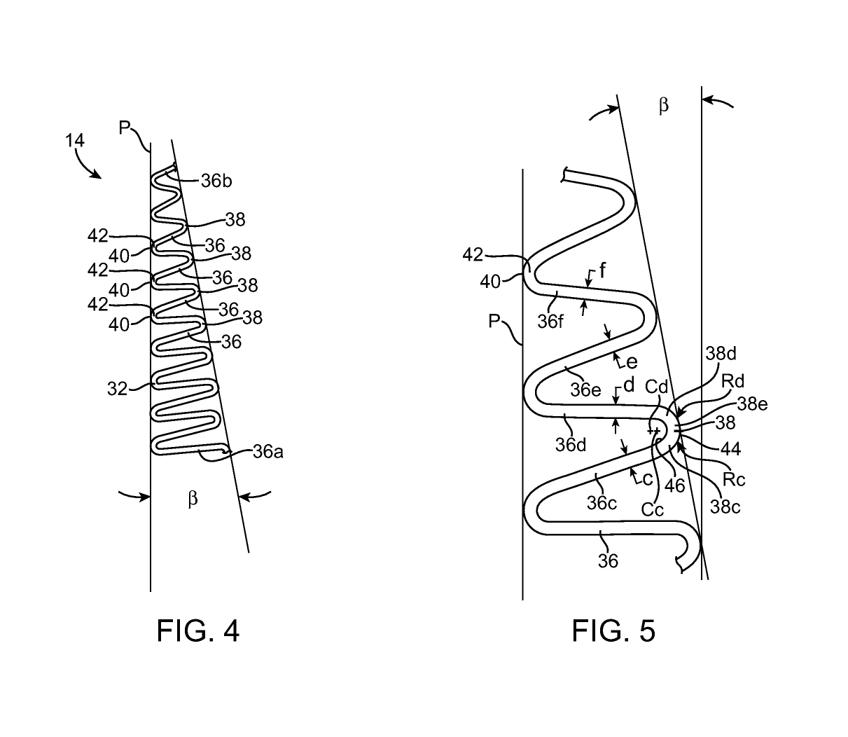

FIG. 4 shows a more detailed view of the first end segment 14 of the stent 10. As illustrated, the end segment 14 is a continuous waveform 32 that is connected at one end to the central portion 12 and is wrapped around the longitudinal axis LA. The continuous waveform 32 includes a plurality of struts 36 and a plurality of crowns 38 that connect adjacent struts, as illustrated in FIG. 4. The waveform 32 is constructed so that a strut 36a at one end of the waveform 32 is longer than any other strut 36 in the waveform 32, and a strut 36b that is at the opposite end of the waveform 32 is shorter than any other strut 36 in the waveform 32. As illustrated, each strut 36 in the waveform 32 has a different length, and the lengths of the struts 36 gradually decrease between the longest strut 36a and the shortest strut 36b, as shown in FIG. 4. This creates a taper having an angle β. Preferably, the angle β of the taper is substantially that same as, or equal to, the pitch angle α of the helix defined by the central portion 12.

The actual lengths of the struts 36 depend on, for example, the desired angle of the taper β of the end segment 14, and are selected so that outer surfaces 40 of end crowns 42, which define one end of the stent 10, are substantially aligned in a single plane P that is substantially perpendicular to the longitudinal axis LA. Such a configuration allows the stent 10 to have an end configuration similar to stents that include a plurality of connected rings that are aligned perpendicularly to the longitudinal axis of the stent.

Similar to the struts 22 of the central portion 12 of the stent described above, by varying the lengths of the struts 36 in the continuous waveform 32 of the first end segment 14, different moments and bending forces may be incurred when the stent 10 deforms radially, such as when the stent 10 is crimped onto a balloon catheter prior to delivery to the targeted site, and/or when the stent 10 is expanded at the site during deployment. To compensate for the different moments and bending forces that are created by the struts 36 having different lengths, the cross-sectional areas of the struts 36 may be varied. For example, as shown in FIG. 4, the longest strut 36a has a greater cross-sectional area (represented by a greater width) than the shortest strut 36b, and the cross-sectional areas of the struts in between the longest strut 36a and the shortest strut 36b are also varied accordingly. As discussed above, the differences in cross-sectional area may be created by altering the width and/or thickness of the strut if the strut has a substantially rectangular cross-section, or altering the diameter of the strut if the strut has a substantially circular cross-section, or altering the dimensions of the minor axis and major axis if the strut has an ellipsoidal cross-section.

The crowns 38 that join the struts 36 may be shaped so that smooth transitions are created between the two different cross-sections of the struts being connected together, while still maintaining the appropriate level of mechanical integrity. For example, as illustrated in FIG. 5, a longer strut 36c has a width ‘c’ that is wider than a width ‘d’ of an adjacent shorter strut 36d, i.e., c>d. The crown 38 that connects the longer strut 36c and the shorter strut 36d is shaped so that the width of the portion of the crown 38 that is connected to the longer strut 36c, represented by 38c in FIG. 5, has substantially the same width (c) as the longer strut 36c. Similarly, the portion of the crown 38 that is connected to the shorter strut 36d, represented by 38d, has substantially the same width as the width (d) of the shorter strut 36d. An intermediate portion 38e of the crown 38 that is in between the portions 38c and 38d, has a varying width that gradually transitions from width c to width d.

In an embodiment, the centers of radii of curvatures that define the outer curved surfaces of portions 38c and 38d of the crown 38 may be off-set to create the gradual transition from width c to width d. For example, as illustrated in FIG. 3, the crown 38 includes an outer surface 44 and an inner surface 46. The inner surface is defined by a constant radius. The outer surface 44 is defined by an outer radius R_{c} that has a center of curvature located at point C_{c}, and an outer radius R_{d} that has a center of curvature located at point C_{d}. As shown in FIG. 5, points C_{c} and C_{d} do not coincide and are off-set from one another, so that the crown 38 has a varying width. The specific crown configuration depicted is provided as an example and is not intended to be limiting in any way.

In an embodiment, the first end segment 14 is formed by laser cutting or chemical etching a tube or sheet of material so that the struts 36 and crowns 38 are created with the proper dimensions so that when the first end segment 14 is contracted or expanded, the first end segment 14 behaves substantially uniformly. The first end segment 14 may be connected to the central portion 12 with two connectors 48, such as welds, one at each end of the end segment 14 (see FIG. 1). Additional connectors may also be used to connect the crowns 38 of the end segment 14 to the crowns 24 of the central portion 12.

In an embodiment, the first end segment 14 may formed by laser cutting a tube, and may be welded to a wire that forms the continuous waveform 18 of the central portion 12. In another embodiment, the first end segment 14 may be formed from a wire that has been drawn down to provide the appropriate cross-sectional areas discussed above. The wire may be a continuation of the wire that forms the continuous waveform 18 of the central portion 12 so that the connector 48 is not needed, or the wire may be a separate wire that is connected to the central portion 12 with the connector 48. In embodiments in which the central portion 12 is formed by cutting a tube or sheet of material, the end segment 14 may be formed from a wire, a cut or chemically etched tube, or a cut or chemically etched sheet of material and connected to the central portion with the connectors 48. Different combinations of wires and cut tubes and sheets of materials may be used. The illustrated embodiment is not intended to be limiting in any way.

It should be appreciated that the second end segment 16 may be formed in the same manner as the first end segment 14 and include the same attributes as the first end segment 14, with the exception that the second end segment 16 will be a mirror image of the first end segment 14 since it is located on the opposite end of the helix of the central portion 12 as the first end segment 14. Therefore, details of the second end segment 16 are not described herein.

The first and second end segments 14, 16 may be formed any suitable material, including but not limited to the materials listed above with regard to the central portion 12. The end segments 14, 16 may be formed from the same material as the central portion 12, or may be formed from different materials, both from each other and from the central portion 12.

It will be appreciated that the foregoing specific embodiments have been shown and described for the purpose of this invention and is subject to change without departure from such principles. Therefore, this invention includes all modifications encompassed within a spirit and scope of the following claims.

What is claimed is:

1. A stent comprising:
a central portion having a first waveform, the first waveform being wrapped around a longitudinal axis of the stent at a pitch to define a plurality of helical turns; and
an end segment connected to one end of the central portion, the end segment having a second waveform that includes a plurality of struts and a plurality of crowns, each of the plurality of struts having a different length so that peaks of the crowns that define an end of the stent lie within a plane that is substantially perpendicular to the longitudinal axis,
wherein cross-sectional areas of the struts having different lengths vary so that the struts move substantially uniformly during radial contraction and/or radial expansion of the stent.

2. A stent according to claim 1, wherein the first waveform is formed from a continuous wire.

3. A stent according to claim 2, wherein the second waveform is formed from a continuous wire.

4. A stent according to claim 3, wherein the continuous wire defining the second waveform is an extension of the continuous wire defining the first waveform.

5. A stent according to claim 3, wherein the continuous wire defining the second waveform is welded to the continuous wire defining the first waveform.

6. A stent according to claim 1, wherein the end segment is formed from a tube and is welded to the central portion.

7. A stent according to claim 1, wherein the central portion is formed from a tube.

8. A stent according to claim 1, wherein the central portion and the end segment are formed from a tube.

9. A stent according to claim 1, wherein the first waveform comprises a plurality of struts and a plurality of crowns, and wherein the plurality of crowns are oriented substantially parallel to the longitudinal axis of the stent.

10. A stent according to claim 9, wherein some of the struts of the first waveform are longer than other struts of the first waveform, wherein cross-sectional areas of the longer struts are greater than cross-sectional areas of the other struts of the first waveform so that the struts of the first waveform move substantially uniformly during radial contraction and/or radial expansion of the stent.

11. A stent according to claim 9, wherein some of the struts of the first waveform are shorter than other struts of the first waveform, wherein cross-sectional areas of the shorter struts are less than cross-sectional areas of the other struts so that the struts of the first waveform move substantially uniformly during radial contraction and/or radial expansion of the stent.

12. A stent according to claim 1, further comprising
a second end segment connected to an opposite end of the central portion, the second end segment having a third waveform, the third waveform including a plurality of struts and a plurality of crowns, each of the plurality of struts having a different length so that peaks of the crowns that define a second end of the stent lie within a second plane that is substantially perpendicular to the longitudinal axis,
wherein cross-sectional areas of the struts of the third waveform having different lengths vary so that the struts of the third waveform move substantially uniformly during radial contraction and/or radial expansion of the stent.

13. A stent comprising:
a central portion having a first waveform formed by a continuous wire, the first waveform being wrapped about a longitudinal axis of the stent so as to form a helix;
an end segment having a second waveform formed from a tube or sheet of material, the second waveform including a plurality of struts and a plurality of crowns, each of the plurality of struts having a different length so that peaks of the crowns that define an end of the stent lie within a plane that is substantially perpendicular to the longitudinal axis;
a first connector constructed and arranged to connect a first end of the second waveform to the central portion; and
a second connector constructed and arranged to connect a second of the second waveform to the central portion.

14. A stent according to claim 13, wherein cross-sectional areas of the struts having different lengths vary so that the struts move substantially uniformly during radial contraction and/or radial expansion of the stent.

15. A stent according to claim 14, wherein struts having longer lengths have larger cross-sectional areas than struts having shorter lengths.

16. A stent according to claim 13, further comprising
a second end segment having a third waveform formed from a tube or sheet of material, the third waveform including a plurality of struts and a plurality of crowns, each of the plurality of struts having a different length so that peaks of the crowns that define an end of the stent lie within a plane that is substantially perpendicular to the longitudinal axis;
a third connector constructed and arranged to connect a first end of the third waveform to the central portion at an opposite end of the central portion as the end segment; and
a fourth connector constructed and arranged to connect a second end of the third waveform to the central portion.

17. A method of manufacturing a stent, the method comprising:
forming a first waveform;
wrapping the first waveform around a mandrel at a predetermined pitch to form a helical shape;
forming a second waveform, the second waveform having a plurality of undulations that decrease in amplitude and in cross-sectional area between a first end of the second waveform and a second end of the second waveform; and
connecting the first end of the second waveform to the first waveform.

18. A method according to claim 17, wherein said connecting comprises welding the first end of the second waveform to the first waveform.

19. A method according to claim 17, further comprising:
forming a third waveform, the third waveform having a plurality of undulations that decrease in amplitude from a first end of the third waveform to a second end of the third waveform and decrease in cross-sectional area between the first end of the third waveform and the second end of the third waveform; and
connecting the third waveform to the first waveform at an end opposite the second waveform.

20. A method according to claim 19, wherein said connecting the third waveform to the first waveform comprises welding the third waveform to the first waveform.

21. A method according to claim 17, wherein said forming the first waveform comprises forming a plurality of struts and a plurality of crowns so that some of the struts of the first waveform are longer than other struts of the first waveform, and so that cross-sectional areas of the longer struts are greater than cross-sectional areas of the other struts so that the struts move substantially uniformly throughout the central portion during radial contraction and/or radial expansion of the stent.

22. A method according to claim 17, wherein said forming the first waveform comprises bending a continuous wire into the first waveform.

23. A method of manufacturing a stent, the method comprising:
forming a first waveform and a second waveform from a solid piece of material, the first waveform having a first plurality of undulations disposed about a longitudinal axis at a pitch so as to form a helix, the second waveform being connected to one end of the first waveform and having a second plurality of undulations that decrease in amplitude and in cross-sectional area between a first end of the second waveform and a second end of the second waveform.

24. A method according to claim 23, wherein the solid piece of material comprises a tube.

25. A method according to claim 24, wherein said forming comprises laser cutting the tube.

26. A method according to claim 24, wherein said forming comprises chemical etching the tube.

27. A method according to claim 23, wherein the solid piece of material comprises a sheet of metal, said method further comprising rolling the sheet of metal into a tube.

28. A method according to claim 27, wherein said forming comprises laser cutting the metal.

29. A method according to claim 27, wherein said forming comprises chemical etching the metal.
